(19) United States
(12) Patent Application Publication
(10) Pub. No.: US 2007/0123923 A1
(43) Pub. Date: May 31, 2007

(54) Implantable medical device minimizing rotation and dislocation

(76) Inventors: Curtis C. Lindstrom, Roseville, MN (US); Ronald W. Heil JR., Roseville, MN (US); Jason A. Shiroff, Minneapolis, MN (US); Paul A. Haefner, Circle Pines, MN (US); Adam W. Cates, Minneapolis, MN (US); Darrell O. Wagner, Isanti, MN (US)

Correspondence Address: SCHWEGMAN, LUNDBERG, WOESSNER & KLUTH, P.A., P.O. BOX 2938, MINNEAPOLIS, MN, 55402, US

(21) Appl. No.: 11/290,355

(22) Filed: Nov. 30, 2005

(51) Int. Cl. A61N 1/375; A61F 2/04

(52) U.S. Cl. 606/192; 623/23.64; 607/36

(57) ABSTRACT

Housings for implantable medical devices are configured so as to engage with surrounding tissues inside of a body and resist both rotation in place and movement through the body. Device housings include shapes, surface features, and/or standard surfaces and may include attached implements that engage the surrounding tissue. Shapes include elongated members, flared ends, and so forth. Surface features include pores, grooves, through-holes, and so forth. Attached implements include needles, barbs, tension springs, and so forth. Also provided are methods for engaging an implantable medical device with surrounding tissues.

### BACKGROUND OF THE INVENTION

Conventional implantable medical devices typically have regular, curved outer surfaces. In FIG. 1, the conventional implantable medical device 101 (i.e. pacemaker) having leads 102 provides an example of the shape of such a device. Some implantable medical devices have smooth biocompatible surfaces in order to prevent metabolization by a host body. Having a smooth, regular outer surface, however, does not ensure a stable position in the body. Devices may (1) rotate, (2) revolve, and/or (3) migrate to a different and unintended position. Such movement can lead to compromised functionality, particularly for subcutaneous defibrillation systems that rely on the device for sensing or providing electrical therapy to the heart.

One example of undesired device displacement is referred to as Twiddler's syndrome, where repeated rotation of a subcutaneous pacemaker causes looping of the pacing wires or catheters, potentially causing poor contact between the wires or catheters and the tissue they are intended to monitor and/or stimulate. FIG. 2 illustrates a normal placement of a conventional subcutaneous pacemaker 101 in a human body. The device sits under the skin with leads 102 which feed through major blood vessels into the heart 203, sensing and/or stimulating heart tissues 204.

FIG. 3 illustrates more closely the area of detail from FIG. 2, including device 101 and leads 102, and FIG. 4 illustrates the same area of detail showing one possible result of Twiddler's syndrome. Here, pacemaker 101 has rotated in place, wrapping leads 102 around itself. Over time, the tension on the leads may result in a poor connection with the tissues of the heart 204.

Presently, implantable devices are sutured or stapled to surrounding tissues in order to prevent dislocation. In general, such acute fixation means have proven to be adequate for most devices. However, in a significant number of cases, failure of the acute fixation means occurs, leaving the implanted device to “float” within the body, as described above. Alternative, chronic means for maintaining device location are needed in these cases. For proper functioning of implantable medical devices, it is desired that the original device orientation and position be maintained throughout the life of the device, without the potential for failure associated with acute fixation means.

### BRIEF SUMMARY OF THE INVENTION

Device housings and methods are provided which minimize rotation and displacement of medical devices implanted within humans and other animals. Device housings include housing shapes, surface features, and/or attached implements which help bind a device to the surrounding tissues. Some embodiments work with a body's natural healing process, enabling encapsulating tissues to anchor the device in place. Additional embodiments engage surrounding tissues directly, either through manual activation, or through automatic activation.

### BRIEF DESCRIPTION OF THE DRAWINGS

The foregoing brief summary of the invention, as well as the following detailed description, is better understood when read in conjunction with the accompanying drawings, which are included by way of example, and not by way of limitation with regard to the claimed invention. In the accompanying drawings, the same or similar elements are labeled with the same reference numbers.

FIG. 1 is a prior art example of a conventional implantable medical device.

FIG. 2 illustrates a prior art example of a normal placement of a conventional subcutaneous pacemaker in a human body.

FIG. 3 illustrates more closely the area of detail from prior art FIG. 2, including a device and attached leads.

FIG. 4 illustrates more closely the area of detail from prior art FIG. 2 showing one possible result of Twiddler's syndrome.

FIG. 5 depicts an example of an implantable medical device situated within a human body in accordance with one or more embodiments.

FIG. 6 depicts a perspective view of a device and the degrees of motion that are impeded by the shape of the device in accordance with one or more embodiments.

FIGS. 7, 8, and 9 depict perspective views of three embodiments having elongated members and areas around which tissue may grow in accordance with one or more embodiments.

FIGS. 10, 11, 12, 13, and 14 depict plan views of five embodiments created using overlapping two-dimensional shapes in accordance with one or more embodiments.

FIGS. 15, 16, and 17 depict perspective views of three embodiments having notched portions in accordance with one or more embodiments.

FIGS. 18, 19, and 20 depict perspective views of three embodiments having members with flared ends in accordance with one or more embodiments.

FIG. 21 depicts a plan view of an optional enclosure for an implantable device in accordance with one or more embodiments.

FIG. 22 depicts a perspective view of an implantable device having several holes or pores in accordance with one or more embodiments.

FIGS. 23 and 24 depict cross-sectional views of a pore on the device of FIG. 22 in accordance with one or more embodiments.

FIG. 25 depicts a perspective view of an implantable device having several modified pores in accordance with one or more embodiments.

FIGS. 26 and 27 depict cross-sectional views of a pore on the device of FIG. 25 in accordance with one or more embodiments.

FIG. 28 depicts a perspective view of an implantable device having anchor structures in accordance with one or more embodiments.

FIGS. 29 and 30 depict cross-sectional views of a portion of the surface of the device from FIG. 28 in accordance with one or more embodiments.

FIG. 31 depicts a perspective view of an implantable device having a mesh anchor in accordance with one or more embodiments.

FIGS. 32 and 33 depict cross-sectional views of a portion of the surface of the device of FIG. 31 in accordance with one or more embodiments.

FIG. 34 depicts a perspective view of an implantable device having through-holes in accordance with one or more embodiments.

FIGS. 35 and 36 depict cross-sectional views of a portion of the device of FIG. 34 in accordance with one or more embodiments.

FIGS. 37 and 38 depict perspective views of an implantable medical device having a rotation implement in accordance with one or more embodiments.

FIGS. 39-41 depict cross-sectional views of a spring attachment abutting and grasping tissue in accordance with one or more embodiments.

FIGS. 42-44 depict side views of an elastic attachment abutting and grasping tissue in accordance with one or more embodiments.

FIGS. 45-47 depict cross-sectional views of an expandable slitted attachment abutting and grasping tissue in accordance with one or more embodiments.

FIGS. 48-49 depict side views of an orthogonal slitted attachment in accordance with one or more embodiments.

FIGS. 50-52 depict cross-sectional views of an inflatable grasper abutting and grasping tissue in accordance with one or more embodiments.

FIGS. 53-56 depict cross-sectional side views of the operation of a capped perforator piercing tissue in accordance with one or more embodiments.

FIGS. 57-59 depict cross-sectional views of the operation of a buried sharp stylet piercing tissue in accordance with one or more embodiments.

FIGS. 60-62 depict cross-sectional views of the operation of an alternative buried stylet piercing tissue in accordance with one or more embodiments.

FIGS. 63-65 depict cross-sectional views of the operation of a “pop rivet” affixing to tissue in accordance with one or more embodiments.

FIGS. 66-68 depict cross-sectional views of the operation of an axial clasp grasping tissue in accordance with one or more embodiments.

FIGS. 69-70 depict cross-sectional views of the operation of a barbed clip affixing to tissue in accordance with one or more embodiments.

FIG. 71 depicts a perspective view of rotating barbs for use in affixing a tube to tissue in accordance with one or more embodiments to tissue.

FIGS. 72 and 73 depict cross-sectional views of a tension spring affixing to tissue in accordance with one or more embodiments.

FIGS. 74-77 depict cross-sectional views of additional deployed tension spring embodiments in accordance with one or more embodiments.

FIGS. 78 and 79 depict cross-sectional views of curved barbs in accordance with one or more embodiments.

FIGS. 80 and 81 depict cross-sectional views of a tension spring extending into tissue in accordance with one or more embodiments.

FIGS. 82 and 83 depict cross-sectional views of a tension spring embodiment extending into tissue in accordance with one or more embodiments.

FIGS. 84 and 85 depict perspective views of an implantable device having retractable helices in accordance with one or more embodiments.

FIGS. 86 and 87 depict perspective views of an implantable device having heat-activated blades in accordance with one or more embodiments.

FIG. 88 depicts a perspective view of an implantable device having curved needles in accordance with one or more embodiments.

FIG. 89 is a flow chart depicting a method for affixing an implantable medical device to surrounding tissue in accordance with one or more embodiments.

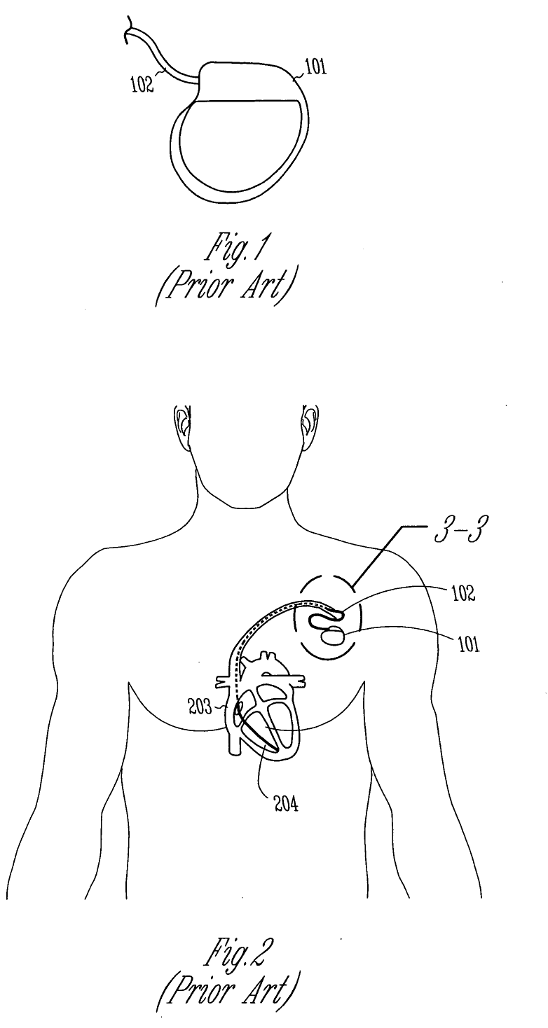

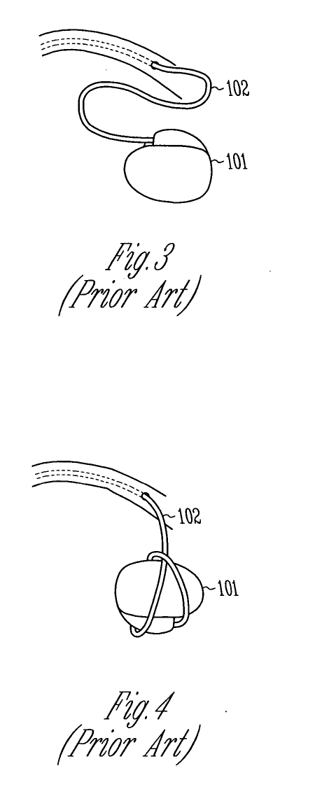

### DETAILED DESCRIPTION OF EMBODIMENTS

Upon insertion of a foreign object into the bodies of humans (and other animals), steps in the natural healing response are triggered which may bring about the elimination of the foreign object via tissue encapsulation. This process will occur whether the insertion is regarded as harmful (e.g., bullets, slivers) or as beneficial (e.g., chronic medical devices). For such encapsulation, the outside of the tissue capsule or pocket is typically anchored to the surrounding native tissue structure and supplied by its vasculature. The inside of the tissue pocket is typically lined with reactive cells (e.g., macrophages, foreign body giant cells, fibroblasts) that attempt to metabolize as well as encapsulate a foreign object.

The housings for implantable medical devices, leads, and catheters may be fashioned using shapes, surface features and/or attached implements that resist movement within a host body using more reliable chronic fixation means. Such housing designs may work with a body's encapsulation process such that encapsulating tissues naturally anchor or affix themselves to the device. Alternatively, these housing designs may be affixed to surrounding tissues using attached implements. Over time, the positional stability created by acute fixation means (e.g., staples or sutures) may be naturally replaced by or enhanced by the chronic fixation means described below.

FIG. 5 depicts an example of an implantable medical device 501 situated within a human body. As previously described, conventional devices may have a tendency to rotate or shift within the body. However, device 501 has been shaped to resist dislocation and rotation within the host body. FIG. 6 depicts a perspective view of device 501 along with the degrees of motion that are impeded by the shape of the device. Because device 501 includes elongated members 602 and 603 at angles to each other, device 501 will resist rotation as well as vertical and horizontal displacement, unlike conventional rounded device 101.

Generally, a device having elongated members extending at angles approaching ninety degrees from each other may resist motion due to greater surface area in a given direction of motion. The longer the members, the greater the moment required to rotate the device, and the greater the force required to shift the device, depending on the direction of motion.

In addition, a device having elongated members 602 and 603 may take advantage of tissue encapsulation, which is a part of the body's natural healing process. As tissues grow around the device and affix themselves to surrounding tissue, the encapsulating tissues serve to anchor the device. For conventional devices, tissue growth around the device does not necessarily prevent motion, since tissues may slide around and/or off a device. For device 501, tissue that grows around member 603 may impede device motion in the vertical direction, and likewise, tissue that grows around member 602 may impede device motion in the horizontal direction.

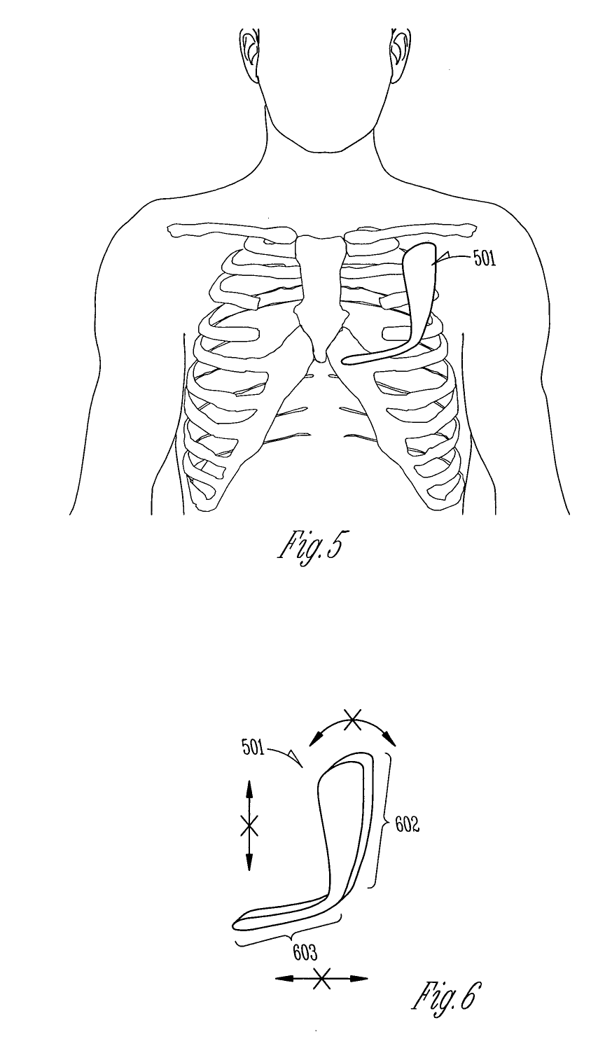

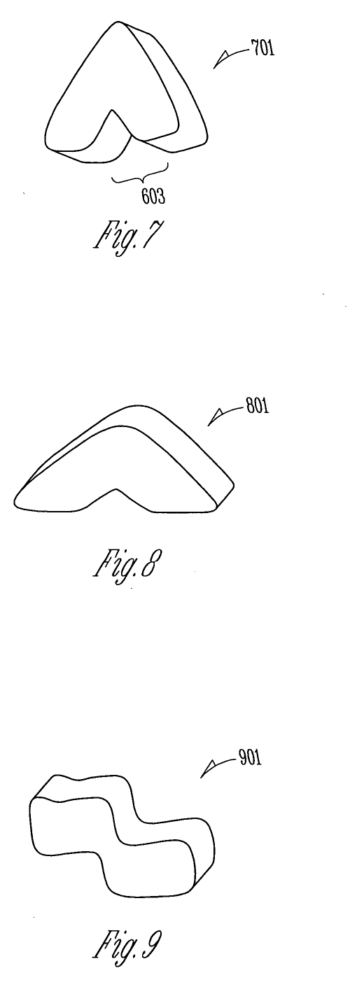

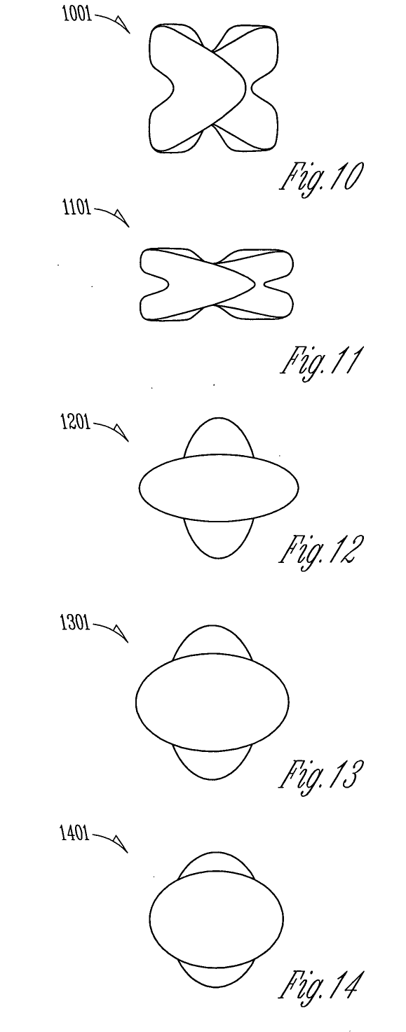

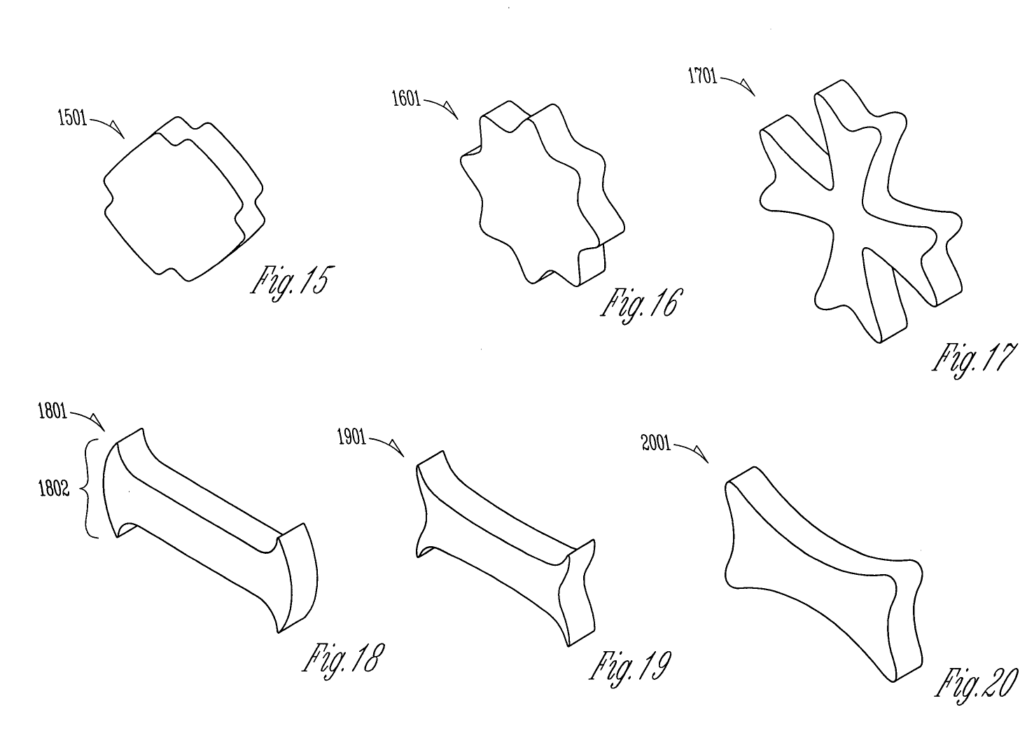

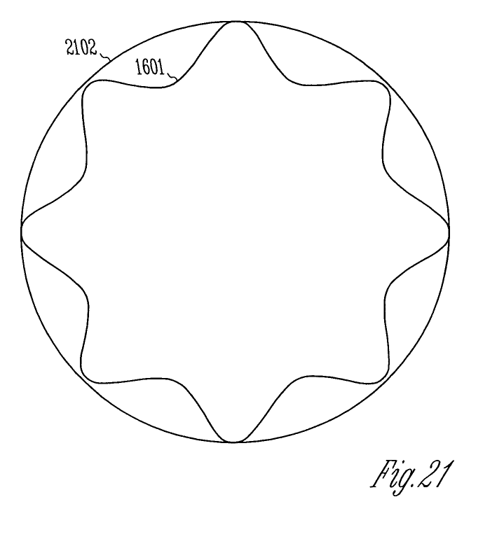

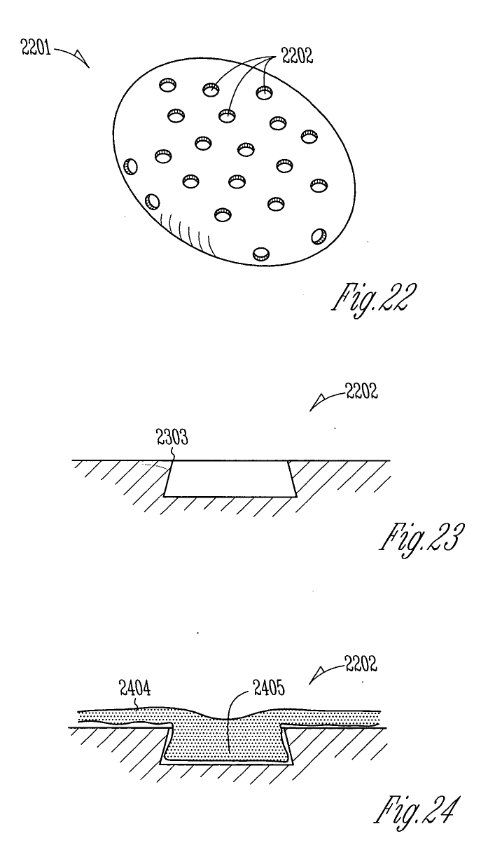

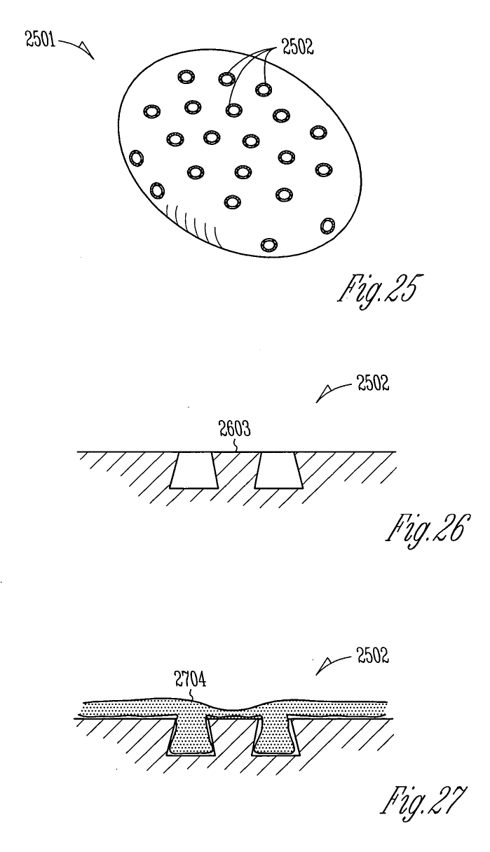

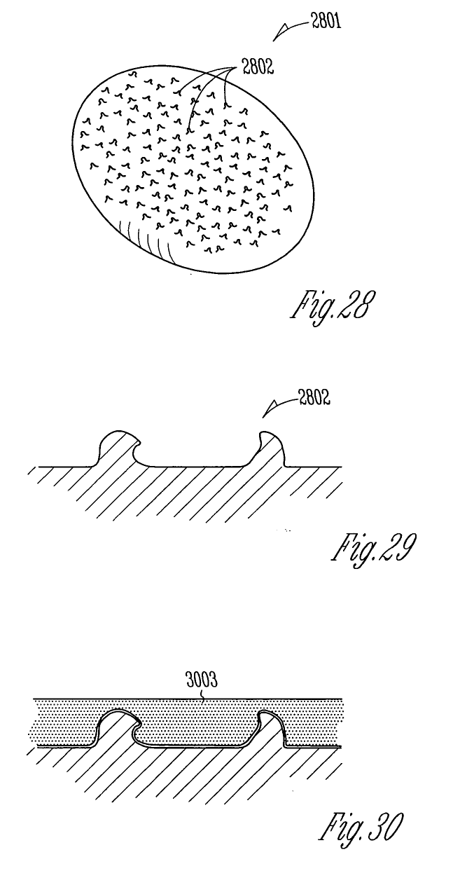

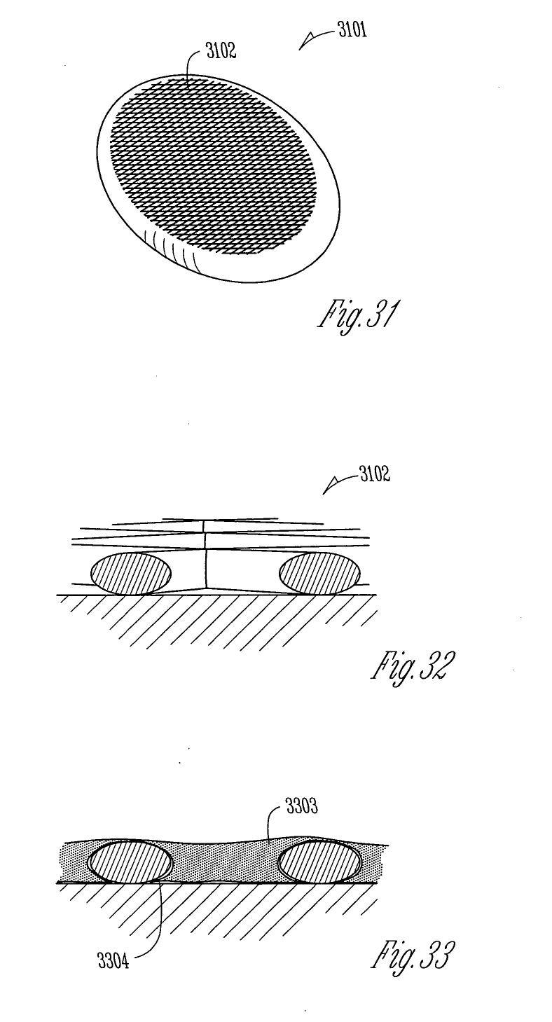

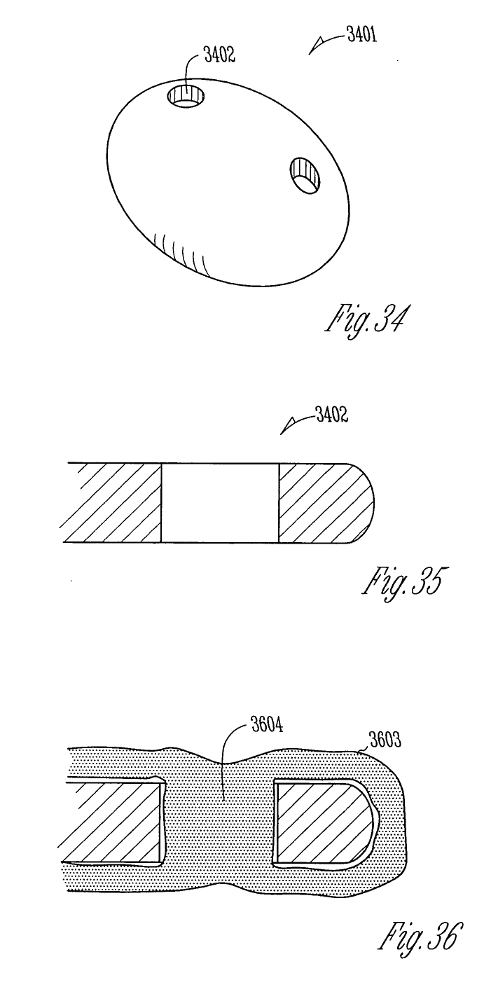

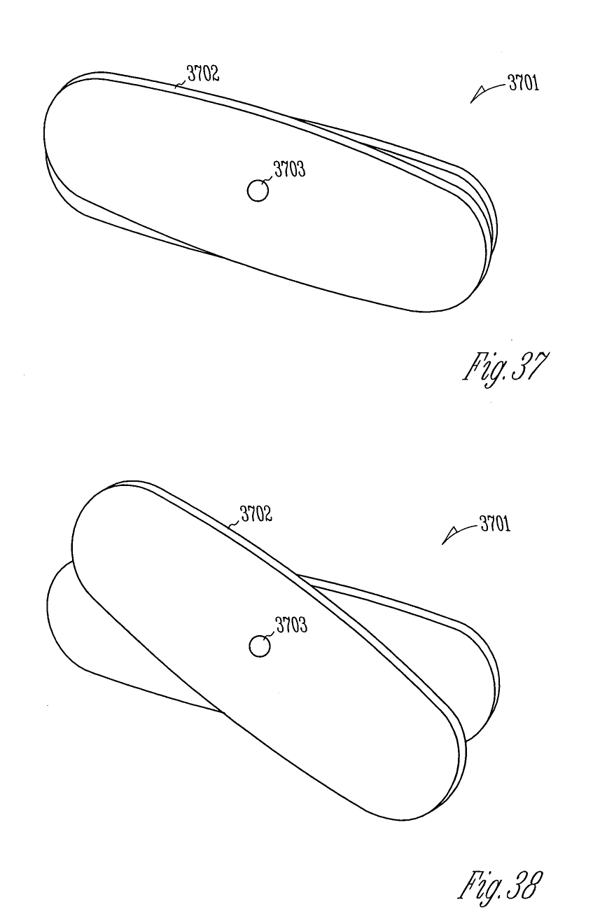

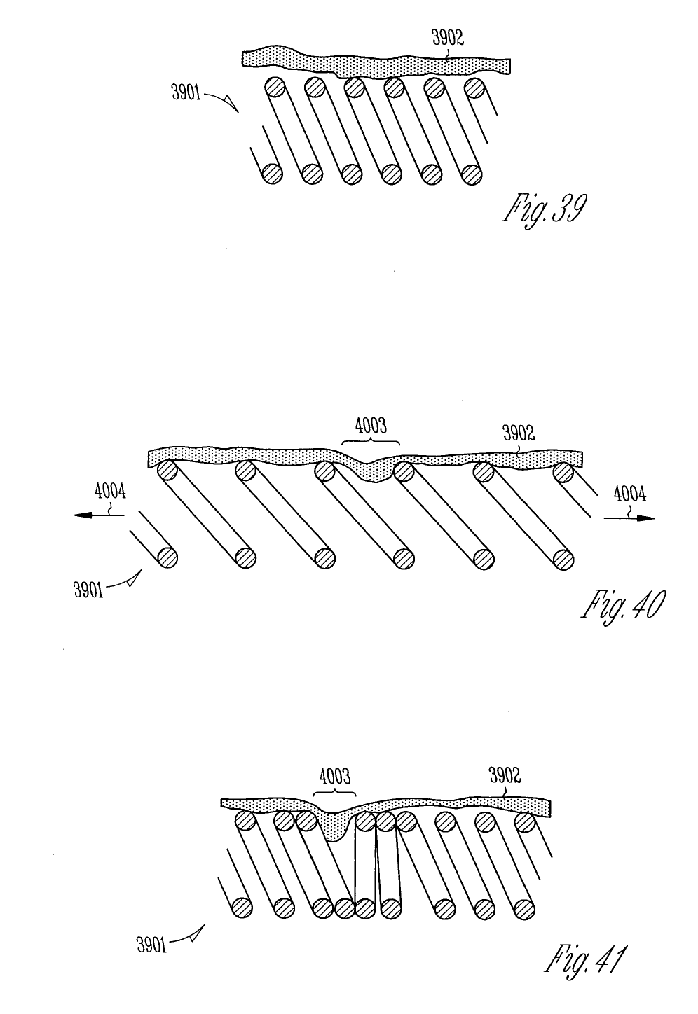

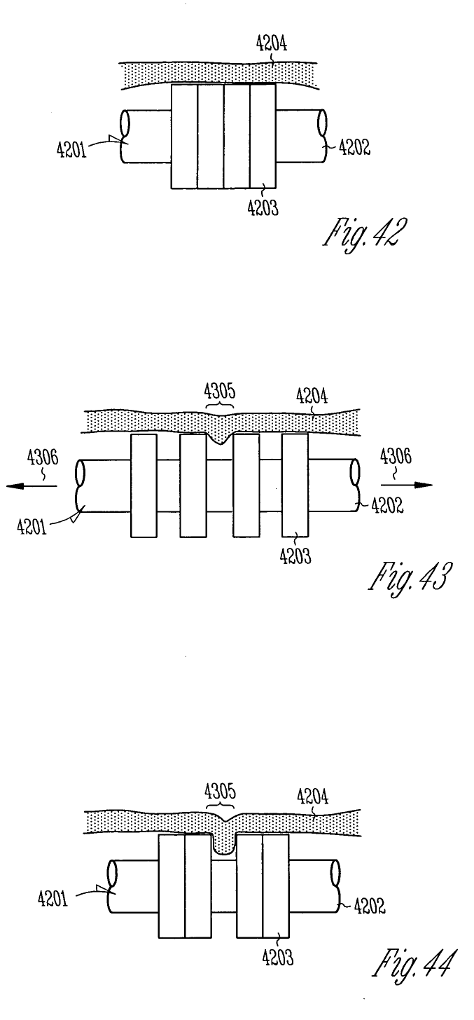

FIGS. 7, 8, and 9 depict perspective views of three embodiments 701, 801, and 901 which include elongated members and areas around which tissue may grow without easily sliding off. Each includes either a notched or narrow portion (e.g., notch 702), once again, where encapsulating tissue may completely surround the device, anchor it to the surrounding tissues, and not easily be loosened from the device.

FIGS. 10, 11, 12, 13, and 14 depict plan views of five embodiments 1001, 1101, 1201, 1301, and 1401 created using overlapping two-dimensional shapes. As before, notched or narrow portions provide constrained areas around which encapsulating tissue may grow and anchor the devices. Even the minimal notches of device 1401, designed using overlapping ovals, provide better stabilization than conventionally shaped devices.

It should be noted that the selection of any particular shape may be made based on any number of considerations, including the size and shape of components housed within the device, the area of the body into which the device is to be placed, the size of the incision in through which the device is to be implanted, cost of materials, type of materials used, and so forth.

FIGS. 15, 16, and 17 depict perspective views of three embodiments 1501, 1601, and 1701 which include notched portions. Device 1701 in particular uses both elongated members and notched portions. As with the embodiments above, the notched portions and elongated members provide locations around which encapsulating tissue may grow and anchor the devices.

FIGS. 18, 19, and 20 depict perspective views of three embodiments 1801, 1901, and 2001 which include elongated members with flared ends. Similar to a notched portion, an elongated member with flared ends (e.g., flared end 1802) allows tissue to grow around the device. The flared ends help prevent the device from sliding beyond the grip of the encapsulating tissues.

FIG. 21 depicts a plan view of an optional enclosure 2102 for an implantable device 1601. Whereas providing notches, elongated members, and flared ends may prevent an implantable device from shifting or rotating in place, those same elements may make placement of a device more difficult. For example, the notches of implantable device 1601 may get caught on skin, fascia, or other tissues when inserted into the body. Providing separable enclosure 2102 may simplify this procedure by covering the notches and easing insertion. Once inserted, enclosure 2102 may be removed.

Enclosure 2102 may additionally be comprised of an absorbable substance to dissolve over a period of time. Examples of such a substance include mannitol, and polyethylene glycol (“PEG”). Other nontoxic, biocompatible, water-soluble materials may be utilized. The enclosure substance may further include a pharmacological agent to enhance healing and/or reduce discomfort associated with the insertion procedure.

Additional embodiments may unevenly distribute weight throughout an implantable device. Such a configuration may assist in positioning a device by using gravity to automatically orient and stabilize the orientation. For example, a circular implantable device may include a heavy weight placed along one edge of the circle. When inserting the device into a body, the weighted portion of the circle may gravitationally bias the orientation of the device into a preferred position. Similar alteration of weight and density in an implantable device may achieve similar results in other device shapes.

In addition to (or instead of) elongated members, notches, flared ends, and other movement-avoiding body shapes, implantable devices may be provided with surface features that help prevent rotation and dislocation of the device. These surface features may work with a body's natural tissue encapsulation process where the tissue grows around the surface features and subsequently becomes anchored to the implantable device.

FIG. 22 depicts a perspective view of a generic implantable device 2201 having several holes or pores 2202. These pores may serve as anchoring locations into which fibrous tissue may grow and become affixed. Although depicted as small circles or ovals here, other pore shapes and sizes may work just as well. The pores may be distributed evenly over the entire device, or may be placed in strategic locations on only one or more sides, in order to maximize anchoring without unduly complicating device removal. Pore location may be strategically selected based on knowledge of likely directions in which the device may shift or rotate. If too many tissue anchors are attached to a device, removing the device may require more time and or pain to the patient.

FIGS. 23 and 24 depict cross-sectional views of pore 2202 on device 2201. Such pores (also referred to as blind holes) may be designed with an overhanging lip 2303, such that tissue 2404 may grow into the pore and become wedged in place. When device 2201 attempts to shift or rotate, tissue 2404, which may be anchored both to device 2201 and to other body tissues, prevents movement due to both the wedge shape of the anchor 2405 and the suction created by the anchor inside the pore. Depending on the angle of the lip 2303, removing tissue from around the device (such as when the device needs to be replaced), may be more or less difficult. Suction alone may be enough to hold anchor 2405 in place, obviating the need for a lip, and possibly simplifying manufacture of the device surface.

The inner walls of pore 2202 may further be textured rather than smooth. The textured character of the walls may be needed so as to provide numerous locations for the fibrotic ingrowth to form anchor attachments to device 2201.

FIG. 25 depicts a perspective view of a generic implantable device 2501 having several modified pores 2502. The modifications made to the pores are made clear in FIGS. 26 and 27, which depict a cross-sectional views of pore 2502. Here, the pore has been modified to include column 2603, which provides additional gripping surface around which fibrous tissue 2704 may grow. Following device implantation, the initial, immature tissue encapsulation will first be produced about the entire device, including within the pores. With the further passage of time, the encapsulation material (i.e. collagen) may undergo natural remodeling and condensation thereby bringing about contraction. Contraction of the tissue around column 2603 further increases the grasping of the device by the tissue.

It should be noted that column 2603 need not be any particular height. Although shown in FIGS. 26 and 27 as being the same height as the surface of a device, such surface features may be shorter (below surface level) or taller (above surface level). Moreover, multiple columns 2603 may vary from location to location, shorter in some and taller in others.

As with device 2201, the pores 2502 may take on different shapes and sizes. Furthermore, a porous structure is not required to take advantage of the enhanced grasping caused by tissue contraction. The exterior of a device may be provided with several grooves or contours generally superficial and tangential to the surface of the device. These grooves should, as with column 2603, form closed loop-like shapes, around which tissue may grow and then contract.

FIG. 28 depicts a perspective view of a generic implantable device 2801 having anchor structures 2802. Anchor structures 2802 are convex to the surface of the device, as opposed to the concave pores of devices 2201 and 2501. These structures may be created as a part of the original housing, or added after initial manufacture, perhaps using an attachment method such as screws, or through sintering of a porous material. FIGS. 29 and 30 depict cross-sectional views of the surface of device 2801, including two anchor structures 2802. Upon insertion, tissue 3003 grows around anchors 2802, creating a bond between the two. As with the columns 2603 of device 2501, tissue 3003 may contract around anchors 2802 over time, further strengthening the bond.

Other surface structures may additionally provide surfaces around which encapsulating tissue may attach itself. FIG. 31 depicts a perspective view of a generic implantable device 3101 having mesh anchor 3102. The mesh 3102 may be metallic, polymeric, fabric, etc., in nature. FIGS. 32 and 33 depict cross-sectional views of device 3101 having mesh 3102. Tissue 3303 may encapsulate device 3101 and once again surround the mesh anchor 3102, creating wedges 3304 similar to those of device 2201. The tissue may further contract around the mesh over time, strengthening its grasp of the device. Other similar porous or mesh structures may similarly induce the desired anchoring effect from encapsulating tissue.

FIG. 34 depicts a perspective view of a generic implantable device 3401 having through-holes 3402. Through-holes 3402 are formed merely by creating holes in the housing of implantable device 3401. FIGS. 35 and 36 depict cross-sectional views of device 3401 having through-hole 3402. Once inserted into a body, the holes may become filled with cellular agents that form numerous fibrous tissue bridges 3604 through the device that connect to the tissue pocket 3603 at both openings. Tissue bridges 3604 through device 3401, especially if situated in multiple through-holes 3402, prevent movement and rotation within the body. Although through-holes 3402 are depicted as circles or ovals, other shapes and sizes may work just as well. However, a tnrough-hole which is too narrow may result in a tissue bridge which is either too brittle, or non-existent.

As discussed above with regard to pores 2202, each of the surface features may benefit from the addition of textured surfaces that provide additional grasping sites for tissue ingrowth. For example, through-holes 3402 may include a rough or otherwise textured surface to provide additional grasping locations for tissue bridges 3604.

The above surface features may be combined with each other, creating hybrid forms and strengths of tissue anchoring. For example, the sintered structures may be combined with the pores to maximize anchoring. Alternatively, a device may include a radial elongated member or arm extending outwards (not shown), the radial arm having a through-hole or other anchoring surface feature. This arm may provide both resistance to shifting and rotating, as well as provide a through-hole through which a tissue bridge may anchor the device. When the device needs to be replaced, the radial arm can merely be broken or detached, and the device removed.

The above mentioned mesh or porous structures, through-holes, blind holes, or grooves may be filled with a biocompatible, inert, water-soluble agent that temporarily fills the structure to generate a smooth surface which may facilitate implantation. Shortly after implantation, the water-soluble agent will dissolve to expose the anchoring means provided. The water-soluble agent may be specially selected to promote the production of tissue encapsulation.

The implantable devices described thus far have included devices designed to passively anchor themselves to surrounding tissues and to resist movement. Additional embodiments may provide active means for affixing a device to surrounding tissues. Such embodiments may require upon implantation and placement the manual activation of one or more implements attached to the device. Alternatively, automatic activation of such anchoring implements may also be utilized. Thus far, examples of implantable medical devices have included defibrillators, but catheters, leads, and other implantable devices may also take advantage of the embodiments and concepts described.

FIGS. 37 and 38 depict perspective views of implantable medical device 3701 having rotation implement 3702. Such a device may be a part of a larger device, and may vary in both size and shape. In FIG. 37, device 3701 is depicted in an inactivated state. This configuration allows easier insertion since no notches or elongated members are exposed to catch on tissues. Once inserted, however, rotation implement 3702 can be rotated around axis 3703 so as to create both notches and elongated members, as shown in FIG. 38. Similar to the passive embodiments described above, these notches and/or elongated members provide anchoring points around which encapsulating tissue may grow, assisting in the stabilization of device 3701.

FIG. 39 depicts a cross-sectional view of an inactive spring attachment 3901 abutting tissue 3902. Spring attachment 3901 may be attached to the outer housing of an implantable medical device (e.g., a pacemaker or associated lead) and can be activated once the device has been implanted. FIG. 40 depicts a cross-sectional view of the same spring attachment 3901 now in an activated state. Here, spring 3901 has been stretched, either by exerting force in the directions of force arrows 4004, or by twisting the spring by applying a moment at one or both ends. Once activated, portions of tissue 3902 may become lodged between widening adjacent coils of spring 3901, such as tissue section 4003. Once the force or moment has been removed, as shown in FIG. 41, spring 3901 relaxes, catching tissue section 4003 in the gap between narrowing coils, effectively grasping the tissue. It is possible that multiple sections of tissue 3902 could be grasped between multiple coils of spring 3901, creating an even stronger bond between the medical device and surrounding tissue.

FIG. 42 depicts a side view of an inactive elastic attachment 4201 abutting tissue 4204. Elastic attachment 4201 includes an elastic sleeve 4202 having a multitude of gripping bands 4203, and may be attached or incorporated into the exterior of an implantable medical device. Elastic sleeve 4202 may be composed of silicone or some other polymeric substance. In FIG. 43, elastic attachment 4201 has been activated through the application of force at the ends of sleeve 4202 in the direction of force lines 4306. When activated, gripping bands 4203 are separated from each other along the axis of the sleeve. Abutting tissue, such as tissue section 4305, may become trapped in the gaps created. Once elastic attachment 4201 is again relaxed, as shown in FIG. 44, tissue is trapped between the gripping bands, holding an associated device in place.

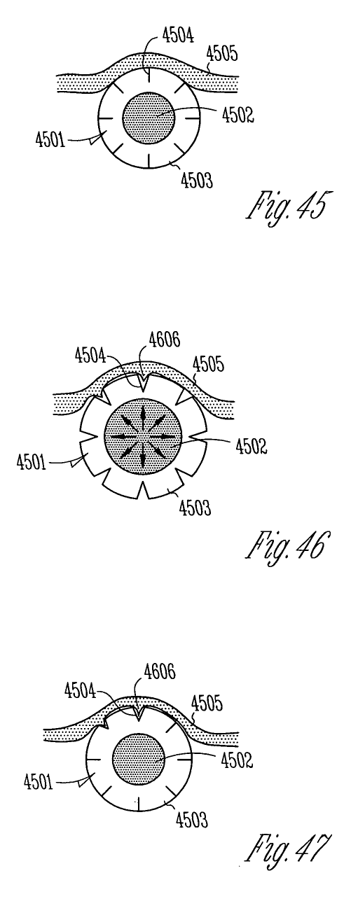

FIG. 45 depicts a cross-sectional view of an expandable slitted attachment 4501 abutting tissue 4505. Such an attachment may be affixed to the exterior of a larger implantable device, such as a pacemaker, or may be integrated into the outer portion of a catheter or lead. Slitted attachment 4501 is composed of inflatable bladder 4502, and elastic ring 4503 having slits 4504. It should be noted that the shape used here is merely representative, and other shapes may work just as well. FIG. 46 depicts the same slitted attachment 4501, although with inflatable bladder 4502 fully inflated, such that elastic ring 4503 has distended, opening slits 4504. As the slits open, portions 4606 of abutting tissue 4505 may enter the openings. Once inflatable bladder 4502 has been deflated, as in FIG. 47, the tissue portions 4606 are grasped by the open slits 4504 of elastic ring 4503.

FIG. 48 depicts a side view of an orthogonal slitted attachment 4801. As with expandable slitted attachment 4501, orthogonal slitted attachment 4801 may be affixed to the exterior of a device, or integrated into the outer surface of a catheter or lead. Orthogonal slitted attachment 4801 includes a multitude of slitted regions 4802. Here, the slits are made in a simple “X” pattern, but other patterns are available. In FIG. 49, as with slitted attachment 4501, the internal pressure of attachment 4801 is increased using an inflatable bladder (not shown) or some other inflation method. The outer surface of the attachment is distended, and slits 4802 expand as shown. Once the internal pressure is returned to normal, slits 4802 return to normal size, grasping abutting tissue (not shown) with the corners created by the pattern.

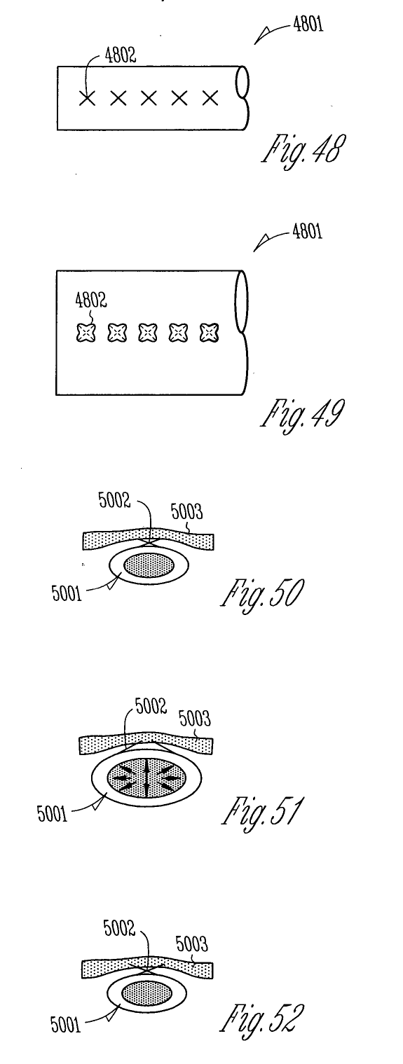

FIG. 50 depicts a cross-sectional view of an inactive inflatable grasper 5001 abutting tissue 5003. Inflatable grasper 5001 includes grasping barbs 5002, which are crossed when the grasper is an inactive state. Grasper 5001 is inflated, increasing its circumference, and separating the grasping barbs 5002 as shown in FIG. 51. Once the expansion of grasper 5001 stops, grasping barbs 5002 pierce tissue 5003. In FIG. 52, pressure within grasper 5001 has returned to normal, and grasping barbs 5002 have returned to their crossed position, pulling tissue 5003 down and locking the tissue in place. As with other attachment implements, alternative shapes and sizes of graspers may be used. Furthermore, increasing the circumference of grasper 5001 may be accomplished using other means, such as inserting a member into the center of the grasper which widens the diameter.

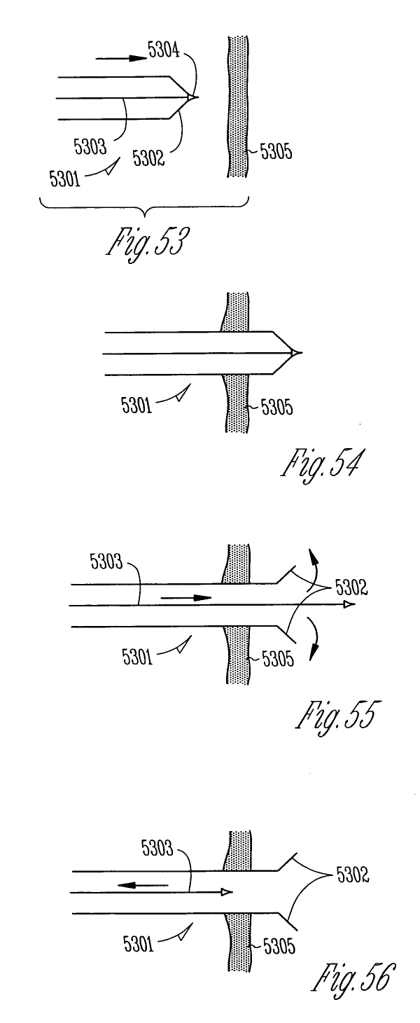

FIGS. 53-56 depict cross-sectional side views of the operation of a capped perforator 5301 piercing tissue 5305. FIG. 53 depicts capped perforator 5301, which is composed of spring-loaded elements 5302, held in a retracted position by a restraining rod 5303, capped by a pointed cap 5304. Such a perforator may be affixed to the housing of a medical device, or to the end of a catheter or lead and used to semi-permanently affix the device, catheter, or lead to tissue 5305. In FIG. 54, capped perforator 5301 has pierced tissue 5305. In FIG. 55, restraining rod 5303 is thrust forward, releasing spring-loaded elements 5302 from under pointed cap 5304. In FIG. 56, restraining rod 5303 is retracted, leaving capped perforator 5301 in place. The wedge created by spring-loaded elements 5302 helps prevent capped perforator 5301 from dislodging from tissue 5305.

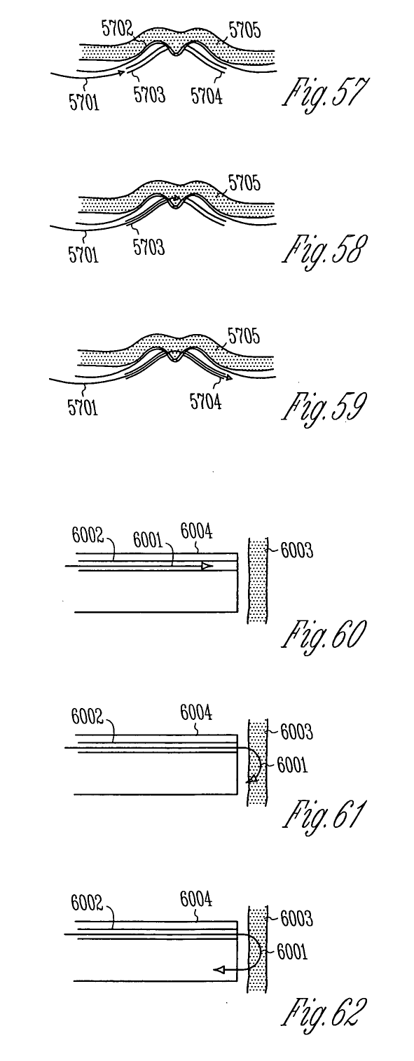

FIGS. 57-59 depict cross-sectional views of the operation of a buried sharp stylet 5701 piercing tissue 5705. FIG. 57 depicts a retracted position for sharp stylet 5701. Outer surface 5702 is formed in specialized “pucker” formation, around which tissue 5705 is pressed. Beneath outer surface 5702, sending tunnel 5703 and receiving tunnel 5704 are formed with metal or otherwise protected interior surface. In FIG. 58, sharp stylet 5701 is advanced through sending tunnel 5703 and into tissue 5705. In. FIG. 59, sharp stylet 5701 continues on through receiving tunnel 5704. Additional buried stylets, or additional pucker formations using the same stylet, may be used to enhance the grasping effect. Alternative formations may be used which place tissue in proximity to a retractable stylus.

FIGS. 60-62 depict cross-sectional views of the operation of an alternative buried stylet 6001 piercing tissue 6003. The operation, which begins in FIG. 60, is similar to that for buried stylet 5701, except that a curved sharp stylet 6001 is advanced through tunnel 6002. Although depicted as being utilized at the end of a cylindrical housing 6004, such a fixation means could be used on the outer housing of implantable devices, including pacemakers, catheters and leads. In FIG. 61, when curved stylet 6001 departs tunnel 6002, it arcs around and back towards the device, piercing and grasping tissue 6003 en route. In FIG. 62, curved stylet advances back into the device, either to be received into a second tunnel (not shown) or possibly to embed itself in a malleable surface such as silicone.

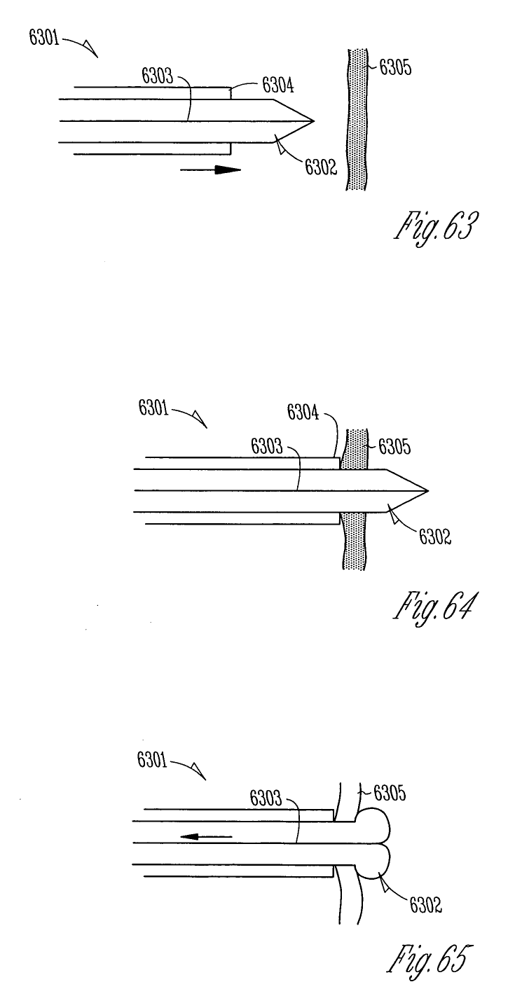

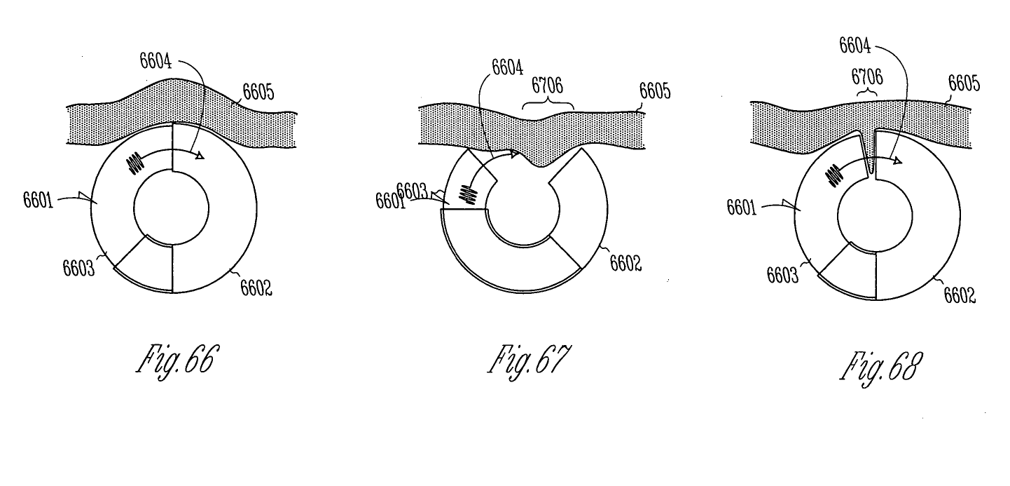

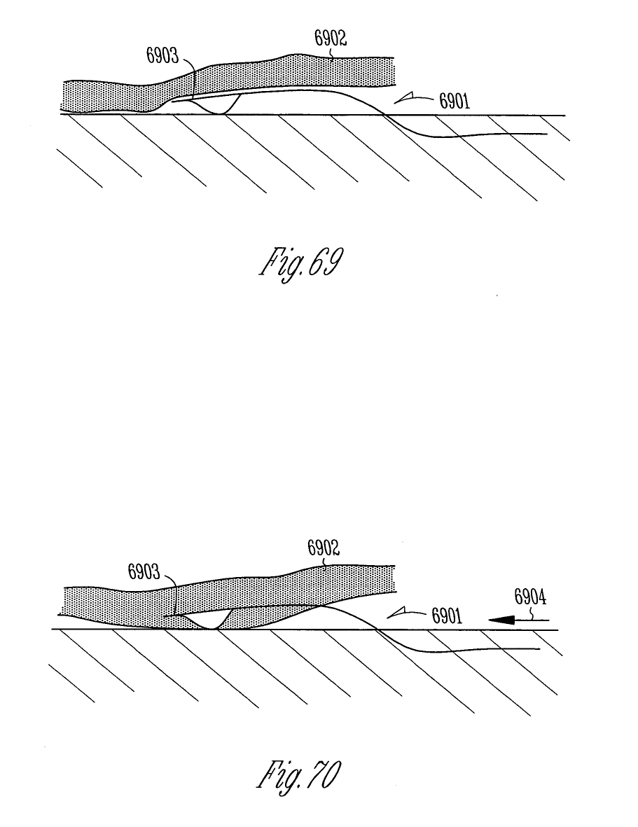

FIGS. 63-65 depict cross-sectional views of the operation of a “pop rivet” 6301 affixing to tissue 6305. Affixing pop rivet 6301 to tissue 6305 is similar to the process of perforating cap 5301. In FIG. 63, pop rivet 6301, consisting of piercing head 6302, stylet 6303, and shoulder 6304, is advanced towards tissue 6305. In FIG. 64, the piercing head advances through tissue 6305, up to shoulder 6304. Finally, in FIG. 65, stylet 6303 is retracted, modifying the shape of piercing head 6302 so that it assumes a “mushroom cap” shape which helps prevent the removal of pop rivet 6301. Using a pop rivet embodiment, the stylet may later be advanced, reforming piercing head 6302, and allowing removal and repositioning of pop rivet 6301 and its associated device.

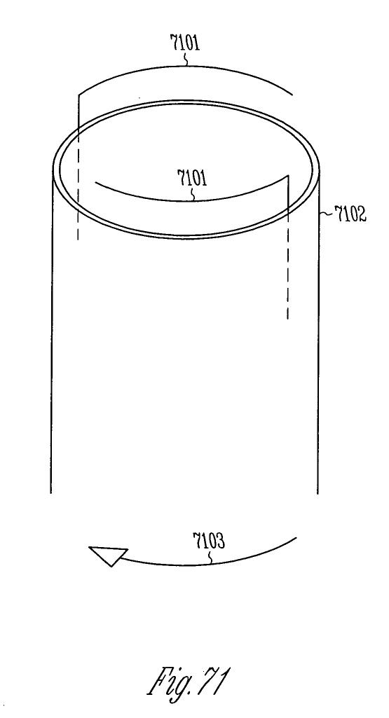

FIGS. 66-68 depict cross-sectional views of the operation of a rotary clasp 6601 grasping tissue 6605. Such a clasp may be incorporated into the outer housing of an implantable device, including a pacemaker, a catheter, or a lead. FIG. 66 depicts clasp 6601 in its relaxed initial position, abutting tissue 6605. Clasp 6601 includes clasp members 6602 and 6603, one of which includes sharp stylet 6604. In FIG. 67, when clasp 6601 is opened while abutting tissue 6605, sharp stylet 6604 is exposed, and a tissue section 6706 enters the gap. When the clasp is again closed in FIG. 68, sharp stylet 6604 pierces tissue section 6706, and relaxing clasp members 6602 and 6603 squeeze and grip the tissue section. Once closed, the clasp both grasps and pierces the tissue section, creating a strong bond between device and tissue.

FIGS. 69-70 depict cross-sectional views of the operation of barbed clip 6901 affixing to tissue 6902. Barbed clip 6901 functions in much the same way as a pen clip. In FIG. 69, barbed clip 6901 includes sharp barb 6903, and is abutted by tissue 6902. In FIG. 70, as the device to which sharp barb 6901 is attached is moved in the direction of arrow 6904, tissue 6902 is pierced, and barb 6903 holds the tissue in place. Multiple barbs may be incorporated into a device surface to further secure device position.

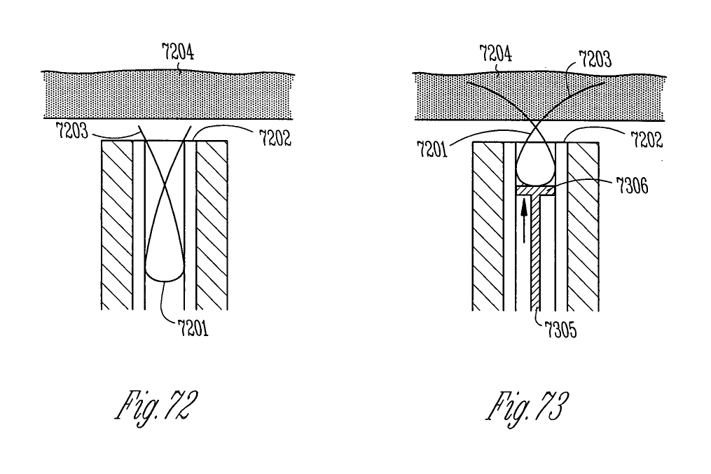

FIG. 71 depicts a perspective view of rotating barbs 7101 for use in affixing tube 7102 to tissue. Tube 7102, which may be attached to any device (e.g., a catheter), can be placed gently against tissue and turned in the direction of arrow 7103, securing rotating barbs 7101 into the tissue. Although two barbs are pictured here, additional barbs may be utilized.

FIGS. 72 and 73 depict cross-sectional views of tension spring 7201 affixing to tissue 7204. FIG. 72 depicts an undeployed tension spring 7201 having curved extensions 7203 placed inside a rigid tube 7202 (made of e.g., glass, metal, or rigid biocompatible polymer). In FIG. 73, tension spring 7201 is deployed, piercing tissue 7204. Deploying the spring may be accomplished by forcing the spring out of tube 7202 using rod 7305. Rod 7305 may terminate or interact with plunger 7306. Rod 7305 may otherwise be provided a groove or slot to guide its progression and interaction with tension spring 7201. Other rod configurations may aid stable deployment of the spring. A tension spring such as the embodiment shown here may be used in conjunction with any type of implantable device, including pacemakers, catheters, leads, and so forth.

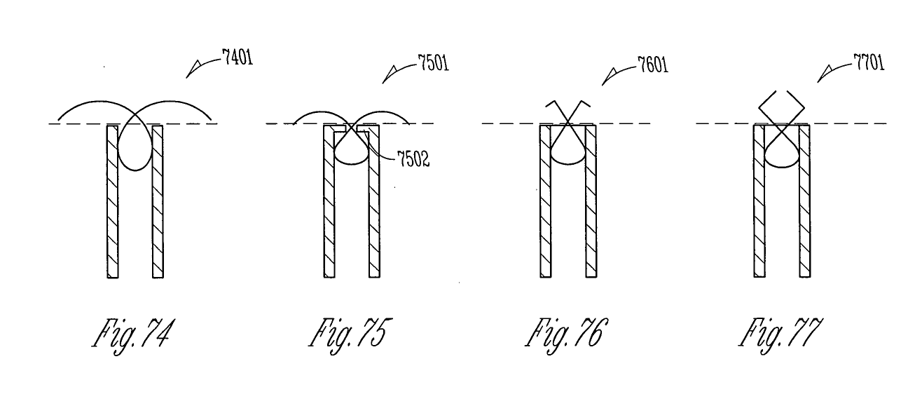

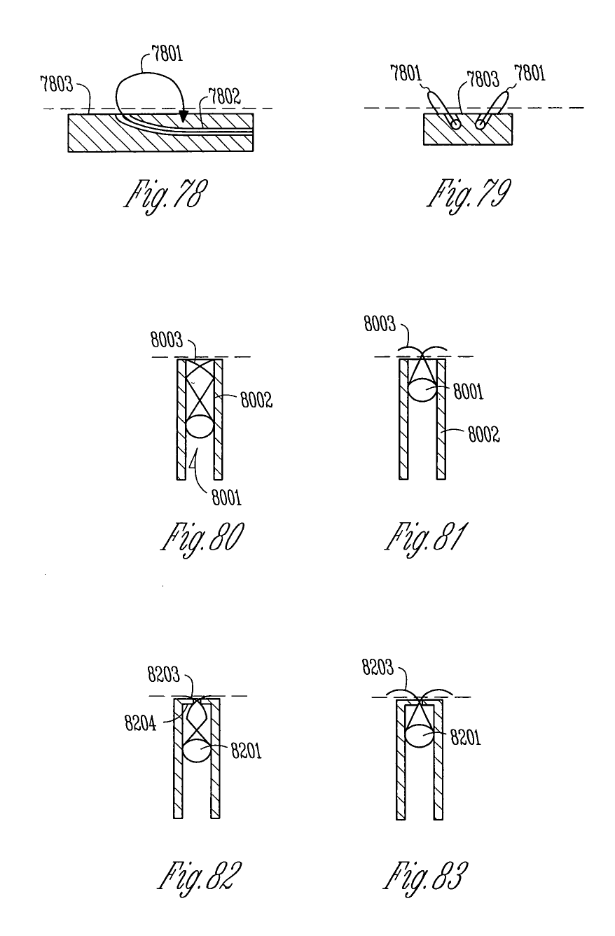

FIGS. 74-77 depict cross-sectional views of additional deployed tension spring embodiments. Spring 7401 employs curved extensions having a tighter curve. Spring 7501 employs a smaller tube opening 7502, causing a shallower, more-controlled tissue entry. Springs 7601 and 7701 both employ barbed or crooked extensions that lodge themselves into tissue.

FIGS. 78 and 79 depict differing cross-sectional views of curved sharp implements 7801, similar to buried stylet 6001. FIG. 78 depicts a side cross-sectional view curved implements 7801. Before the implements are extended (not shown), they initially sit buried in tunnel 7802 within device 7803. Once device 7803 is in place, an implement 7801 is extended by forcing it through tunnel 7802. The implements are curved such that they will curl through adjacent tissue and arc back into device 7803. FIG. 79 depicts a front cross-sectional view of implements 7801. The sharp implements may be angled away from each other as shown in order to spread the attachment points with the tissue. Sharp implements 7801 may include barbs to hinder removal. Alternatively, smooth sharp endings may facilitate retraction of the implements if needed.

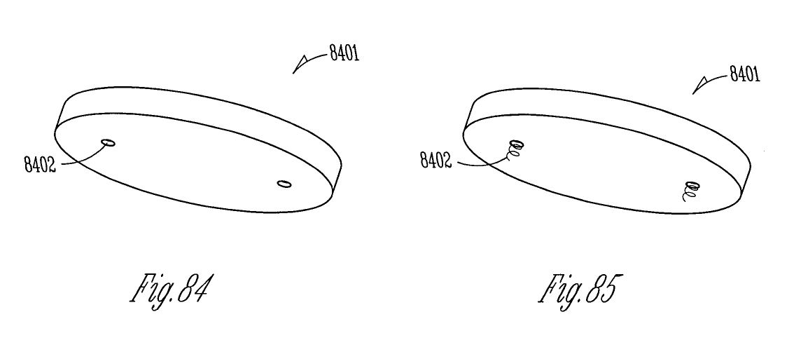

FIGS. 80 and 81 depict cross-sectional views of tension spring 8001 extending into tissue. Similar to previously described tension springs, in FIG. 80, spring 8001 lies in a tense or wound up state within tunnel 8002 until the associated device is placed. Once placed, spring 8001 is extended into the surrounding tissue as shown in FIG. 81, and spring arms 8003 unfold and extend broadly into the tissue.

FIGS. 82 and 83 also depict cross-sectional views of a separate tension spring embodiment 8201 extending into tissue. As shown in FIG. 82, the opening 8204 through which tension spring 8201 extends is narrowed. This may cause spring arms 8203 to embed themselves in the tissue at a shallower level, as can be seen in FIG. 83. It also may allow the movement and extension of spring arms 8203 to be more controlled and deliberate.

FIGS. 84 and 85 depict perspective views of a generic implantable device 8401 having retractable helices 8402. FIG. 84 shows retractable helices 8402 completely retracted, which is the position they would be in while device 8401 is being placed within a patient. Once placed, a doctor may extend helices 8402 (e.g., by rotating them from the opposite side) as shown in FIG. 85. Helices 8402 twist into the underlying tissues (e.g., fascia) and become attached, similar to a corkscrew. If device 8401 needs to be replaced at a later date, helices 8402 may be unscrewed from surrounding tissues, and the device removed. Although two helices are shown, additional helices may be used.

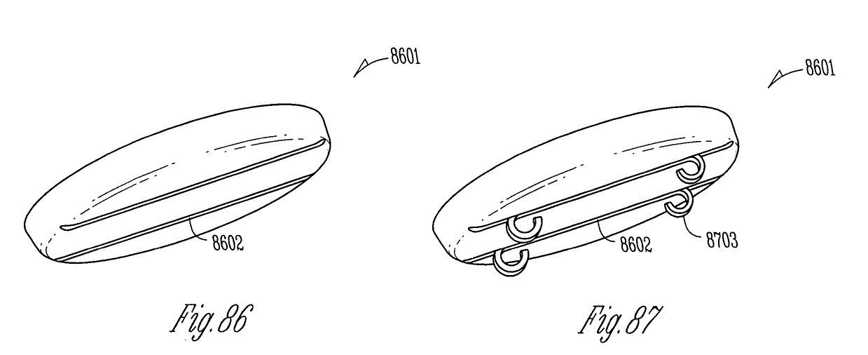

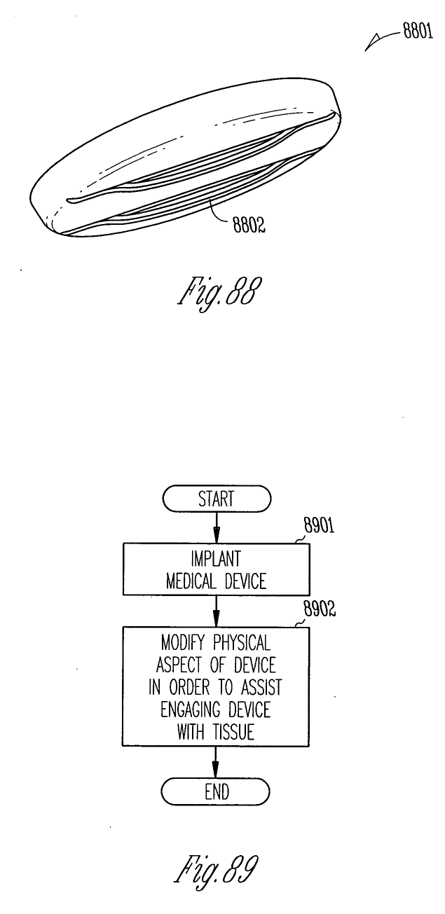

FIGS. 86 and 87 depict perspective views of a generic implantable device 8601 having heat-activated blades 8602. FIG. 86 shows heat-activated blades 8602 when they are in their initial inactive state. Blades 8602 may be manufactured using a heat-activated substance, one that takes a new shape when heated. For example, the blades may be manufactured using a nickel-titanium alloy (e.g., nitinol) which, when heated, returns to a predetermined shape. FIG. 87 depicts blades 8602 after placement in a patient, when they have been warmed and reshaped into a predetermined curl. The curls 8703 grasp surrounding tissues, holding device 8601 in place.

FIG. 88 depicts a perspective view of a generic implantable device 8801 having curved needles 8802. The curved needles 8802 displayed here, similar to previously described stylets and barbs, can be threaded through device 8801 after placement within a patient. Alternatively, needles 8802 may be formed with a heat-activated substance as described above, a substance that bows upon entry into a warm body.

FIG. 89 is a flow chart depicting a method for affixing an implantable medical device to surrounding tissue. At step 8901, a medical device is implanted in a host body, whether it is a catheter, a lead, a pacemaker, and so forth. At step 8902, a physical aspect of the medical device is modified, enabling the device to engage with the surrounding tissue. Different types of modified physical aspects have been previously described.

It should be noted that the devices and methods described above are not limited to use with human patients. Other animals may benefit from preventing displacement of implantable medical devices.

While devices and methods embodying the present invention are shown by way of example, it will be understood that the invention is not limited to these embodiments. The devices and housings described are merely examples of the invention, the limits of which are set forth in the claims which follow. Those skilled in the art may make modifications, particularly in light of the foregoing teachings. For example, each of the elements of the aforementioned embodiments may be utilized alone or in combination with elements of the other embodiments.

We claim:

1. A housing for an implantable medical device, wherein the housing is configured so as to prevent rotation and displacement of the device within a host body.

2. The housing of claim 1, wherein an exterior surface of the housing is configured to include features which resist movement of the housing within the host body.

3. The housing of claim 2, wherein the features comprise one or more concave pores into which tissues may be naturally generated.

4. The housing of claim 3, wherein an inner portion of a concave pore includes a non-smooth surface texture.

5. The housing of claim 2, wherein the features comprise one or more through-holes through which tissue bridges may be naturally generated.

6. The housing of claim 5, wherein an inner portion of a through-hole includes a non-smooth surface texture.

7. The housing of claim 2, wherein the features comprise one or more flared members around which tissue may be naturally generated.

8. The housing of claim 2, wherein the features comprise one or more sintered anchors around which tissue may be naturally generated.

9. The housing of claim 2, wherein the features comprise a plurality of elongated members that extend at angles approaching ninety degrees from each other.

10. The housing of claim 2, wherein the features comprise a plurality of notches.

11. The housing of claim 2, wherein the features comprise one or more loop-like raised areas around which tissue may be naturally generated.

12. The housing of claim 2, wherein the features comprise a mesh-like structure around which tissue may be naturally generated.

13. The housing of claim 1, wherein the housing is unequally weighted in order to gravitationally position and maintain the device in a preferred orientation.

14. The housing of claim 1, wherein the configured housing includes one or more implements capable of affixing to surrounding tissues.

15. The housing of claim 14, wherein the one or more implements comprises an implement capable of piercing surrounding tissues.

16. The housing of claim 15, wherein the one or more implements comprises a needle.

17. The housing of claim 15, wherein the one or more implements comprises a barb.

18. The housing of claim 14, wherein the one or more implements comprises an implement capable of grasping surrounding tissues.

19. The housing of claim 18, wherein the one or more implements capable of grasping surrounding tissues are configured to include an active spring that controls the grasping of surrounding tissue.

20. The housing of claim 14, wherein the one or more implements require manual intervention in order to affix the housing to the surrounding tissues.

21. The housing of claim 14, wherein the one or more implements affix the housing to the surrounding tissue without manual intervention.

22. The housing of claim 21, wherein the one or more implements are comprised of a heat-activated substance.

23. The housing of claim 22, wherein the heat-activated substance comprises a nickel-titanium alloy.

24. A method for affixing an implantable medical device to surrounding tissue, the method comprising:
inserting the implantable device into the body of an animal; and
modifying manually a physical aspect of the device upon insertion, whereby the modification assists in affixing the implantable device to surrounding tissue.

25. The method of claim 24, wherein the step of modifying a physical aspect of the device comprises turning a portion of the device.

26. The method of claim 25, wherein turning a portion of the device engages a helix structure with surrounding tissue.

27. The method of claim 25, wherein turning a portion of the device creates notches around which encapsulating tissue may grow.

28. The method of claim 25, wherein turning a portion of the device extends a spring.

29. The method of claim 24, wherein the step of modifying a physical aspect of the device comprises moving the device in a particular direction causing barbs to engage with surrounding tissue.

30. The method of claim 24, wherein the step of modifying a physical aspect of the device comprises pushing a portion of the device.

31. The method of claim 30, wherein pushing a portion of the device comprises advancing a stylet through the device.

32. The method of claim 30, wherein pushing a portion of the device comprises advancing a needle through the device, whereby a portion of the needle engages with surrounding tissue.

33. The method of claim 30, wherein pushing a portion of the device comprises pushing a tension spring through the device, whereby a portion of the tension spring engages with surrounding tissue.

34. The method of claim 24, wherein the step of modifying a physical aspect of the device comprises pulling a portion of the device.

35. The method of claim 34, wherein pulling a portion of the device comprises extending a spring.

36. The method of claim 34, wherein pulling a portion of the device comprises pulling a wire.

37. The method of claim 24, wherein the step of modifying a physical aspect of the device comprises increasing the pressure within a portion of the device.

38. The method of claim 37, wherein increasing the pressure within a portion of the device comprises inflating a portion of the device.

39. The method of claim 37, wherein increasing the pressure within a portion of the device causes a plurality slits on an elastic portion to open.

40. The method of claim 37, wherein the step of modifying a physical aspect of the device further comprises decreasing the pressure within the portion of the device.

41. The method of claim 24, further comprising:
enclosing the implantable device in a separable enclosure prior to inserting the device into the body of the animal.

42. The method of claim 41, wherein the separable enclosure is comprised of a substance absorbable within the body of the animal.

43. A separable enclosure for an implantable medical device, the separable enclosure comprising a material for easing entry of the implantable medical device into a host body.

44. The separable enclosure of claim 43, the separable enclosure being comprised of a substance absorbable by the host body.

45. The separable enclosure of claim 44, wherein the substance is comprised of at least one of mannitol and polyethylene glycol.
